(19) United States
(12) Patent Application Publication
(10) Pub. No.: US 2004/0013735 A1
(43) Pub. Date: Jan. 22, 2004

(54) Method for granulation of active substances by low pressure extrusion to obtain directly compressible granules

(76) Inventors: Stephan Martin-Letellier, Marennes (FR); Jean-Claude Le Thiesse, Saint-Etienne (FR)

Correspondence Address: BURNS DOANE SWECKER & MATHIS L L P, POST OFFICE BOX 1404, ALEXANDRIA, VA, 22313-1404, US

(21) Appl. No.: 10/380,954

(22) PCT Filed: Sep. 21, 2001
(86) PCT No.: PCT/FR01/02948

(30) Foreign Application Priority Data
Sep. 22, 2000 (FR) 00/12112

(51) Int. Cl. A61K 9/14; B27N 3/00

(52) U.S. Cl. 424/489; 264/109

(57) ABSTRACT

The invention concerns a method for formulating one or several active substance(s) in the form of directly compressible granules comprising wet process granulation using a binding solution of said active substance and optionally associated excipients, then drying the resulting granules. The invention is characterised in that said granulation is carried out by low pressure extrusion of the mixture of the active substance(s), binding solution and optionally excipient(s).

The field of the invention is the formulation of active substances, notably pharmaceutically active ingredient(s) into the form of granules suited to compression.

Conventionally, pharmaceutically active ingredients are formulated in the form of tablets. Now, a great many pharmaceutically active substances are not per se made of materials that can be directly compressed. In order to remedy this unsuitability, these active substances are conventionally converted beforehand to give them sufficient plasticity to make them compressible. The most usual pre-conversion is to granulate the active substances.

In general, granulation is a technique that makes it possible to increase the particle size of a powder. Its purpose more specifically is to convert pulverulent solids into aggregates of varying size, varying resistance and varying porosity, which are known as granules. It also allows the granular products to be endowed with practical properties such as, inter alia, reduced propensity to create dust, better flowability, improved dispersibility, greater mixability or better ability to be pelletized. There are three main routes to granulation: the wet route, the molten route and the dry route. Dry granulation is generally favoured for products capable, under stress, of acquiring cohesion between particles, for example by compacting a mix of ingredients using rollers. Molten granulation is generally employed in the case of thermally stable products for which low-porosity granules are desired, for example by flaking molten active ingredients or extruding an active ingredient in suspension in a molten excipient (polymer, fat, etc). Wet granulation, for its part, requires the addition of a solution to the mix of ingredients, the purpose of this solution being to act as a binder so as to agglomerate the individual particles. This agglomeration is obtained by bringing the individual particles closer together by applying mechanical energy and by forming capillary bridges of the binder solution between these individual particles. This third route therefore generally involves performing a subsequent drying step.

The present invention relates more specifically to granulation in a pharmaceutical field using the wet route, and is aimed more specifically at proposing a new way of preparing granules suited to direct compression.

Conventionally, shaping a pulverulent active substance into the appearance of a tablet involves the following steps. First of all, the various active ingredients and excipients are mixed. This mix is then formulated into the state of granules using various technologies such as mixers (high-speed or high-shear), fluidized beds or alternatively atomizers, for example. A subsequent drying step is carried out. In general, the dried granules are then sized. In order to manufacture tablets with the desired properties (mechanical strengths, dissolution dynamics), excipients are added to the granules by mixing and this final mix is introduced into a pelletizer.

The steps performed to obtain products which are ready for pelletizing are therefore numerous and, for industrial viability reasons, it would be advantageous to be able to perform this conversion of pulverulent active substance into granules that can be directly compressed, continuously.

More specifically, the present invention is aimed at a method for formulating one or more active substance(s) into the form of granules that can be directly compressed, comprising wet granulation of the said active substance and, where appropriate, of the associated excipients using a binder solution, followed by the drying of the granules thus obtained, characterized in that the said granulation is carried out by low-pressure extrusion of the mix of active substance(s), binder solution and, where appropriate, excipient (s)

The claimed method is notably advantageous in that it yields granules which can be directly compressed, it being possible for this method to be performed continuously or discontinuously.

According to a preferred variant, the method is carried out continuously.

Apart from the advantages mentioned hereinabove, the present invention makes it possible for any products whose applicability might have been diminished through an accidental drift in the system, for example during the drying step, to be recycled into the process. Typically, the ability of granules to be compressed is dependent upon their residual water content, and the present method precisely allows products which do not have the water content required for their compression to be recycled.

The method claimed is carried out using a low-pressure extruder. The term “low-pressure” is to be understood as meaning a pressure of a few bar, generally lower than 10⁶ Pa (10 bar), typically of the order of 2 to 4×10⁵ Pa and preferably of 3 to 4 10⁵ Pa (3 to 4 bar). This idea of low pressure distinguishes the extrusion at issue here from the conventional extrusion carried out on polymers, which is a molten extrusion carried out at high pressure (several tens of to several hundred bar).

Extruders which are particularly suited to this are dome extruders, basket extruders and radial extruders. The low pressure level obtained in these extruders is associated with the relatively large size of the open area of the extrusion screen and to the formulation forces needed in order to limit the forces exerted on this screen.

Extruders suitable in the framework of the present invention are, preferably, extruders with low shearing power. In this respect, single screw extruders or extruders without screw such as a basket extruder are particularly suitable.

The various constituents of the formulation are mixed beforehand in the presence of a binder solution. This mixing can be carried out using continuous or discontinuous conventional methods. By way of an illustration of these, mention may, in particular, be made of the following techniques: ribbon mixers, high-speed mixers, share mixers, high-shear mixers, single-screw or double-screw continuous mixers.

The binder solution may be produced either from a polymer used in a solvent (generally water) or by mixing in the binding agent in the dry state and adding water to the total mix. The binder solution may be introduced either by pouring it directly into the mix and/or by spraying it. It is generally introduced at ambient temperature, namely between 15° C. and 40° C., but this introduction may be at higher temperatures, of the order of 50° C. to 90° C., depending on the nature of the binder solution in question.

The binder solution is introduced and spread by stirring all the compounds together. This mixing may be carried out in the same mixer as the one used previously or in another mixer and, depending on the apparatus used, a continuous mode or a discontinuous mode may be obtained. In general, the average mixing time is of the order of a few minutes (from 2 to 10 minutes), but a longer mixing time, of the order of 10 to 30 minutes, may prove necessary.

On leaving the mixer, the mix obtained is fed continuously into a low-pressure extruder.

The moisture content of the mixer to be introduced into the extruder is also an important parameter. This is because the moisture both makes the various binder agents present in the mix effective and improves the extrudability of the said mix. It thus makes it easier for the mix to pass through the extrusion screen by giving it a certain plasticity and by improving the lubrication of the system. An excessively low moisture content does not allow the production of sufficiently cohesive extrudates. There is therefore the fear that they might return to dust as soon as they reach the drying step, or even as soon as they leave the extruder. By contrast, an excessively high moisture leads the formation of long rods, which tend to clump together at the extruder outlet.

It is thus particularly advantageous to carry out the extrusion at a moisture content of between 5% and 40% by weight of the amount of dry matter (active substances and excipients to be formulated) involved, preferably between 10% and 30%. Of course, the value of the moisture content that allows good extrusion will need to be adjusted to suit the active ingredients and formulations being studied.

The method of the invention is preferably carried out using a dome extruder, a basket extruder or a radial extruder, preferably using a dome extruder.

A low-pressure dome extruder is an apparatus with a single screw or a twin screw which forces the wet mix to pass through a die situated at the end of the screw. This die is in the shape of a hemisphere or dome, in the case of a single-screw extruder or in the form of two touching hemispheres, in the case of a twin-screw extruder. The particular feature of the extrusion technology considered according to the invention is associated with the fact that the pressure applied in the region of the screen is low.

As regards the parameters of the die, namely the diameter aperture, its aperture ratio (the ratio between the open area and the area of the dome) and its thickness, these are tailored so as to obtain granules with the desired properties: particle size distribution, mixability, and the mechanical and dissolution properties of the resulting tablets.

According to a preferred embodiment of the invention, the die has an aperture with a diameter of between 300 μm and 2 mm and preferably of between 500 μm and 1 mm, an aperture ratio varying from 5% to 75% and preferably from 10% to 60% and a thickness ranging from 0.2 mm to 1.0 mm and preferably from 0.3 mm to 0.8 mm. Generally, a die with multiple apertures is used, for example, with 100 to 1000 apertures.

Of course, the diameter of the die aperture needs to take account of the maximum size of the particles to be extruded, in order to avoid any blockage.

The extrudates obtained are then dried using conventional technologies, for example such as drying on plates or in an oven, drying in a fluidized bed or in a continuous vibrated fluidized bed. The passage through the extrusion screen makes it possible to obtain extrudates of uniform size, hence giving rise to uniform drying and therefore better control over the application properties. Advantageously, the residual moisture content of the granules obtained at the end of the drying step may be controlled to a moisture content which is uniform to with 0.5%. This residual moisture content is of course able to vary according to the active substance that is to be granulated. Thus, in the particular case of paracetamol (N-acetyl-para-aminophenol), the residual moisture content of the granules obtained after drying is preferably equal to 2%±0.5%.

The granules thus obtained may or may not then be sized by forcing them to pass through a mesh. The size of the granules leaving the extruder is therefore not critical, it being possible for the particle size distribution to be adjusted during this later sizing step. A spheronization step subsequent to the extrusion may also be contemplated.

FIG. 1 represents an industrial flow chart for the implementation of an extrusion method according to the present invention with all the specific operations discussed above, in a continuous mode.

The device comprises a mixer 1, fed by a reactor 3 with a binder solution prepared in said reactor, via a pump 5, and also with dry matter (for example in form of granules) contained in a hopper 7, via a screw feeder 9. The mix obtained from the binder solution and the dry matter in the mixer 1 forms a slurry which is fed via a duct 11 into an extruder 13. At the outlet of this extruder, the slimy has the form of very long filaments placed on a vibrating belt 15 within a drying chamber 17. The drying chamber 17 is fed with hot air by means of a heater battery 19, which generates circulated hot air and sent within the drying chamber 17 by means of a fan 21. The drying chamber 17 is equipped with an exhaust means for the cooled air, said means comprises a filter 23 adapted for treating the air at the outlet of the drying chamber 17, the treated air being than evacuated into the atmosphere by means of an exhaust fan 25.

At the outlet of the drying 17, after the path on the vibrating belt 15 from one end to the other of the drying chamber 17, the dried extrudate is extracted from the drying chamber 17 and directed to a calibration means 27 which allows the yield of granules according to the dimensional specifications at the outlet of the device.

The operation of compressing the granules obtained is of course within the competence of those skilled in the art. This involves determining the residual moisture content to be kept in the granules, and choosing the excipients needed for the correct use of the final mix in the pelletizer and for the desired practical properties (standard tablets, effervescent tablets, tablets that disperse in the mouth, tablets with controlled release, etc.).

Generally, any active substance can be converted by the method claimed as long as it proves to be compatible with granulation and appropriate to shaping into the form of tablets.

Thus, all active substances in form of powder with a median particle diameter of about 1 to 100 μm may be efficiently formulated according to the claimed method.

The amount of active substance involved in the compressible pharmaceutic granules prepared according to the method of the present invention can vary widely. More particularly, it is between 0.001% and 99.5% by weight of the total composition, the remainder being made up of the associated excipients.

The method claimed proves to be particularly advantageous in formulating pharmaceutically active substances which require a granulation step prior to pelletizing.

As regards the active substances, these may be of various natures such as for example, pesticides, cosmetics and preferably pharmaceuticals. They may also be nutritional complements containing for example vitamins. By way of illustrative and non-limiting example of these active substances, mention may, in particular, be made of anti-rheumatism agents, anti-inflammatories, analgesics, psychotropic agents, steroids, barbiturates, vasodilators, therapeutic agents targeted at the gastro-intestinal tract, contraceptives, anti-hypertension drugs, cardiovascular or cardio-protective agents.

Representatives of these active substances that may be mentioned in particular are paracetamol (acetyl-para amino-phenol), glycerol gaïacol (3-(2-methoxy phenoxy)-propane 1,2 diol) and ketoprofen (2-(3-benzoylphenyl)propionic acid).

The claimed method is thus particularly advantageous for the preparation of directly compressible granules based on paracetamol.

This active substance is generally introduced in a pulverulent form. However, it may also relate to granules obtained at the end of a granulation of this active substance and which, for various reasons such as an inadequate residual moisture content, for example, are not adapted to the performing of a compression step. In the particular case of paracetamol, this may especially be granules which have a residual moisture content other than 2%±0.5%. In general, this content is too low and in particular lower than 2%±0.5%.

The pharmaceutically active ingredients may be formulated with excipients that make it possible to obtain the desired practical properties of the granules. These excipients may be diluents such as lactose, sucrose, calcium phosphates; cohesive agents, such as hydrophillic polymers like polyvinylpyrrolidone, cellulose, cellulose derivatives (hydroxyproplymethylcellulose, etc), natural, modified natural or synthetic gums (gelatine, carob gums, guar gums, xanthan gums, alginates, carrageenans), native or precooked starches; disintegrating agents such as native starches, superdisintegrators such as sodium starch glycolate; flow agents such as silica, talc; lubricating agents such as stearic acid, magnesium stearate, calcium stearate; preservatives such as potassium sorbate, citric acid, ascorbic acid. All these constituents are generally introduced into the mixer with the active substance that are to be formulated. However, these excipients may be fully or partially incorporated into the binder solution.

More particularly, regarding the binder solution, this is generally based on water or on an aqueous solvent. This binder solution conventionally incorporates a material which, because of its nature, encourages the particles of active substance that are to be formulated to agglomerate to form granules. Binding agents such as polyvinylpyrrolidone, cellulose, cellulose derivatives (hydroxyproplymethylcellulose, hydroxypropylcellulose), natural, modified natural or synthetic gums (gelatine, carob gums, guar gums, xanthan gums, alginates, carrageenans), native or precooked starches are particularly suited to this type of function.

The binder solution is generally used in a content of 5% to 40% by weight of the active substances that are to be formulated. In fact, its quantity varies widely and is, in particular, associated with the characteristics of the ingredients to be formulated (solubility, hygroscopy, particle size distribution, rheology) and with the desired practical properties (mechanical properties, particle size distribution, dissolution dynamics). Adjusting this quantity is within the competence of the person skilled in the art.

The examples are figure which follow are given by way of non-limiting illustration of the present invention.

### FIGURE

FIG. 1: Representation of an industrial flow chart for the implementation of an extrusion method according to the invention present in the continuous version.

### APPARATUS AND METHOD

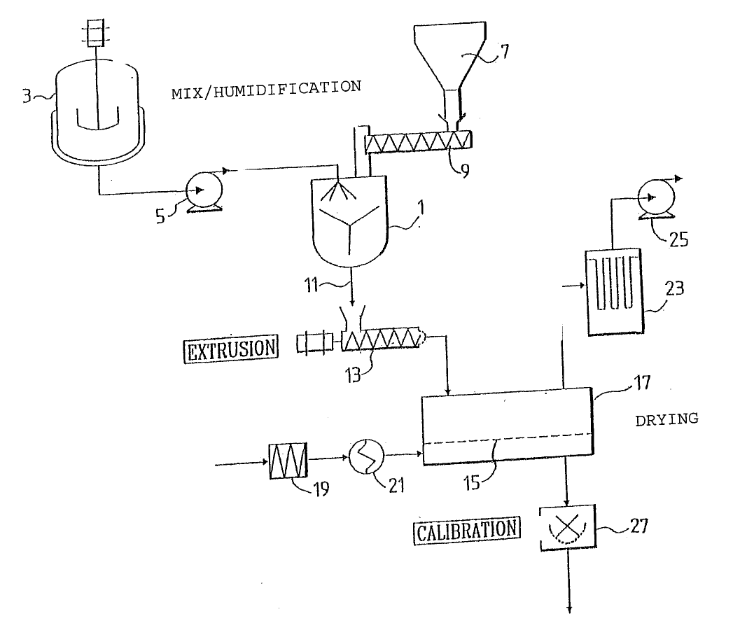

For all the tests set out hereinafter, the procedure was the same. The industrial flow chart is according to the one illustrated in FIG. 1.

The active substance and the excipients used in each formulation tested were weighed then introduced into a high-shear mixer of the Diosna® make, model V25. The powders were mixed dry for five minutes. The binder solution used for all the tests given was water at ambient temperature, introduced into the abovementioned mixer using a dropping funnel. Impregnation with this binder solution was achieved by mixing the system for a residence time that varied from 5 minutes to 30 minutes depending on the formulations involved.

The product thus moistened was then fed by means of a feed hopper with a metering screw into the inlet of the low-pressure extruder. The low-pressure extruder used during the tests was a dome extruder of the Fuji Paudal® make, model DGL-1. It had a single screw and the extruder screen is a hemisphere. The aperture diameter of the extruder screens used varied from 300 μm to 1 mm, for an aperture ratio varying from 12% to 57% and a thickness of 0.3 mm to 0.8 mm. the pressure used is lower than 4 or 3.10⁵.

The extrudates obtained were dried in a fluidized bed of the Retsch® make, model T61, to the desired residual moisture content, namely between 1.0% and 2.5% according to the formulation. As an indication, this may be obtained by heating between about 40 and 50° C. for about 20 minutes. The dry granules were then sized in an apparatus of the Erweka® make, model AR 400, through screens with a mesh diameter of between 350 μm and 1 mm, depending on the target particle size.

The granules thus produced were mixed with an external phase consisting of a lubricant and eventually a flowing agent and a disintegrating agent in a bicone blender of the Retsch® make, model UA1, and were characterized in respect of their ability to make tablets by passing them through a rotary press of the Manesty® make, Betapress model. To quantify the ability of the granules to be compressed, the tablets were evaluated with respect to their mechanical properties and dissolution properties using the standard Pharmacopoeia tests.

In these tests, the desired properties for the tablets were:

maximum friability of the order of 1.0%

minimum cohesion of the order of 1.0 Mpa Further were determined in the case of paracetamol:

compulsory maximum disintegration time 15 minutes (Pharmacopoeia)

a compulsory minimum amount dissolved of 80% in 30 minutes (Pharmacopoeia).

The cohesion was determined from measurements made on the tablets, and is defined as follows:

$$Cohesion = \frac{2 \times hardness \times acceleration due to gravity }{π \times diameter \times thickness}$$

The tests used, as active ingredient, paracetamol or acetyl-para-aminophenol (Rhodapap Pulvérisé Dense NP®, Rhodia), glycerol guaiacol or 3-(2-methoxyphenoxy-propane 1.2-diol (Guaifenesin, Rhodia) and ketoprofen or 2-(3-benzoylphenyl)proprionic acid (Aventis), and excipients such as polyvinylpyrrolidone (Kollidon K30®, 490®, BASF), precooked corn starches (Starch 1500®, Colorcon or Starch 1551®, National Starch),maltodextrine (MD040 Grain processing corp.), a super disintegrant (sodium croscarmellose, Ac-Di-Sol, FMC), silica (Aerosil 200, Degussa) and stearic acid (Stearine TP 1200®, Stéarinerie Dubois). Au used excipients are described in the Handbook of Pharmaceutical Excipients Ed ₂nd A. Wade—P. J. Weller (1994).

### EXAMPLE 1

The following formulation was used for this test:

| Internal phase: | |
|---|---|
| Paracetamol | 90.00% |
| Polyvinylpyrrolidone K30 ® | 0.60% |
| Starch 1500 | 6.40% |
| Ac-Di-Sol | 2.50% |
| External phase: | |
| Stearic acid | 0.50% |

A quantity of 2653.3 g of internal phase, corresponding to a quantity of 2400 g of paracetamol, was introduced into the high-shear mixer. After mixing, a quantity of 800 g of distilled water was introduced into the mixer, namely a moisture content of 30% with respect to the dry mix. After extrusion through a 700 μm die, the product was dried and brought to a residual moisture content of 1.9%, then sized through a 800 μm screen.

After 13.4 g of stearic acid were added, the granules were characterized in compression and their galenic qualities were, measured. The compression was carried out at a rate of 45 000 tablets per hour. The results of these tests are given in table 1 hereinafter. The forces PC and C, expressed in tonnes, are, respectively, the precompression and compression forces applied to the mixtures to be compressed. The dissolution dynamics are expressed here as the time taken to dissolve 80% of the active ingredient.

TABLE I

| | | | | | Disinte- | Dissolution |
|---|---|---|---|---|---|---|
| Forces (t) | | Hardness | Friability | Cohesion | gration | 80% |
| PC | C | (kg) | (%) | (MPa) | time(s) | dissolved |
| 0.0 | 1.5 | 9.84 | 0.23 | 1.23 | 35 | — |
| | 2.0 | 7.88 | 0.54 | 0.96 | 35 | — |
| | 2.5 | 9.83 | 0.90 | 1.14 | 32 | — |
| 0.5 | 1.5 | 12.26 | 0.15 | 1.49 | 63 | — |
| | 2.0 | 14.42 | 0.14 | 1.80 | 63 | 7 min 24 s |
| | 2.5 | 16.50 | 0.12 | 2.11 | 54 | |

The tablets manufactured with or without precompression have satisfactory properties, entirely meeting the desired criteria, whether from the mechanical point of view (friability, cohesion) or from the disintegration and dissolution point of view with good dynamics.

### EXAMPLE 2

A new formulation was used for this test. This was:

| Internal phase: | |
|---|---|
| Paracetamol | 89.70% |
| Polyvinylpyrrolidone | 3.00% |
| Starch 1551 ® | 3.30% |
| Ac-Di-Sol | 2.50% |
| Colloidal silica | 1.00% |
| External phase: | |
| Stearic acid | 0.50% |

A quantity of 2969 g of internal phase, corresponding to a quantity of 2677 g of paracetamol, was introduced into the high-shear mixer. After mixing, a quantity of 742 g of binder solution (distilled water) was introduced into the mixer, namely a moisture content of 25% with respect to the dry mix. After extrusion through a 700 μm die, the product was dried and brought to a residual moisture content of 1.7% and sized through a 370 μm screen.

After 15 g of stearic acid had been added, the granules were characterized in compression and their galenic properties were measured. The tests were carried out at compression rates of 45 000 tablets per hour and 60 000 tablets per hour. The results are given in tables II and III hereinafter, respectively.

TABLE II

| Forces (t) | | Hardness | Friability | Cohesion | Disintegration |
|---|---|---|---|---|---|
| PC | C | (kg) | (%) | (MPa) | time(s) |
| 0.0 | 1.0 | 11.78 | 0.32 | 1.41 | 110 |
| | 1.5 | 15.21 | 0.32 | 1.90 | 100 |
| | 2.0 | 19.11 | 0.12 | 2.41 | 110 |
| | 2.6 | 17.53 | 0.29 | 2.23 | 110 |
| 0.5 | 1.0 | 11.93 | 0.24 | 1.42 | 120 |
| | 1.5 | 16.63 | 0.23 | 2.06 | 100 |
| | 2.0 | 20.04 | 0.15 | 2.53 | 110 |
| | 2.5 | 22.81 | 0.19 | 2.92 | 170 |

TABLE III

| | | | | | Disinte- | Dissolution |
|---|---|---|---|---|---|---|
| Forces (t) | | Hardness | Friability | Cohesion | gration | 80% |
| PC | C | (kg) | (%) | (MPa) | time(s) | dissolved |
| 0.0 | ˜0.6 | 7.78 | 0.77 | 0.94 | 70 | 3 min 0 s |
| | ˜0.8 | 10.11 | 0.26 | 1.22 | 70 | 3 min 0 s |
| | 1.0 | 12.09 | 0.33 | 1.43 | 70 | 4 min 0 s |
| | 1.5 | 15.55 | 0.28 | 1.92 | 90 | |
| | 2.0 | 17.46 | 0.20 | 2.18 | 100 | |
| | 2.5 | 18.21 | 0.51 | 2.27 | 120 | |
| 0.5 | 1.1 | 12.91 | 0.25 | 1.53 | 90 | — |
| | 1.2 | 14.75 | 0.30 | 1.86 | 90 | — |
| | 2.1 | 20.75 | 0.16 | 2.62 | 140 | — |

The properties of the tablets obtained were entirely exceptional from the mechanical point of view, and irrespective of the operating conditions. Under operating conditions which in theory would seem prejudicial (no precompression, very low compression level and high compression rate), highly satisfactory mechanical properties were obtained, with very swift dissolution dynamics.

### EXAMPLE 3

The formulation used in example 1 was used again for this test. A quantity of 4000 g of internal phase was prepared using the same method as in example 1.

After extrusion, a first fraction was, however, initially too dry (residual moisture content of 0.5%). The product was therefore reintroduced into the high-shear mixer and water was added to bring the mix to a moisture content that was acceptable for it to pass on to the extrusion stage. The re-moistened product was then extruded and the subsequent drying was- controlled in such a way as to obtain a final residual moisture content of 1.8%.

The second fraction was, for its part, not dried enough (residual moisture content of 3.6%). The product was once again introduced into the high shear mixer and the moisture content was raised before it was passed back to the extruder. The product extruded once again was dried to a residual moisture content of 1.9%.

The two fractions were kept separately and, after the addition of the external phase as defined in example 1, the granules were evaluated in compression. The results of this evaluation, carried out at a compression rate of 45 000 tablets per hour, are given in tables IV and V hereinafter, for the fractions which initially were too dry and for the fractions which initially were not dry enough, respectively.

TABLE IV

| | | | | | Disinte- | Dissolution |
|---|---|---|---|---|---|---|
| Forces (t) | | Hardness | Friability | Cohesion | gration | 80% |
| PC | C | (kg) | (%) | (MPa) | time(s) | dissolved |
| 0.0 | 1.6 | 15.15 | 0.25 | 1.84 | 145 | — |
| | 2.1 | 16.22 | 0.29 | 1.97 | — | — |
| | 2.4 | 12.25 | 0.70 | 1.52 | — | — |
| 0.5 | 1.5 | 14.70 | 0.26 | 1.83 | — | — |
| | 2.0 | 17.57 | 0.20 | 2.27 | — | 5 min 0 s |
| | 2.4 | 19.64 | 0.22 | 2.56 | 170 | |

TABLE V

| | | | | | Disinte- | Dissolution |
|---|---|---|---|---|---|---|
| Forces (t) | | Hardness | Friability | Cohesion | gration | 80% |
| PC | C | (kg) | (%) | (MPa) | time(s) | dissolved |
| 0.0 | 1.6 | 15.06 | 0.20 | 1.83 | 130 | — |
| | 2.0 | 13.81 | 0.31 | 1.71 | — | — |
| | 2.5 | 11.54 | 0.56 | 1.44 | — | — |
| 0.5 | 1.5 | 14.30 | 0.29 | 1.79 | — | — |
| | 2.1 | 17.15 | 0.21 | 2.20 | — | 5 min 0 s |
| | 2.5 | 19.24 | 0.28 | 2.50 | 170 | |

The tablets obtained during this test all had very satisfactory mechanical properties and the dissolution dynamics measured on the tablets manufactured with a precompression of 0.5 tonne and a compression of 2.0 tonnes were fast. In addition, and irrespective of which fraction is considered, the granules had similar behaviours as regarded their suitability for compression and the properties of the tablets were equivalent. This test therefore shows that it is possible to recycle all products which, in terms of their residual moisture content, are not compliant, into the low-pressure extrusion method described here and that the resulting products have very satisfactory practical properties.

### EXAMPLE 4

During this test, the content of active ingredient was increased to 95%. The following formulation was used:

| Internal phase: | |
|---|---|
| Paracetamol | 95.00% |
| Polyvinylpyrrolidone K90 | 2.23% |
| Starch 1551 | 1.48% |
| Silica | 0.15% |
| External phase: | |
| Ac-Di-Sol | 0.74% |
| Stearic acid | 0.25% |
| Colloidal silica | 0.15% |

A quantity of 1873.1 g of internal phase, corresponding to a quantity of 1800 g of paracetamol, was introduced into a share mixer. After mixing, a quantity of 255.4 g of distilled water was introduced into the mixer, namely a moisture content of 13.6% with respect to the dry mix. After extrusion through a 1 mm die, the product was dried and brought to a residual moisture content of 1.3%, then sized through an 800 μm screen.

After 21.55 9 of external phase had been added, the granules were characterized in compression and their galenic properties were measured. The compression was carried out at a rate of 45 000 tablets per hour. The results of these tests are given in table VI hereinafter.

TABLE VI

| Forces (t) | | Hardness | Friability | Cohesion |
|---|---|---|---|---|
| PC | C | (kg) | (%) | (MPa) |
| 0.0 | 1.7 | 13.30 | 0.23 | 1.65 |
| | 2.1 | 15.61 | 0.28 | 1.98 |
| | 2.5 | 15.26 | 0.29 | 2.01 |
| 0.5 | 1.5 | 12.98 | 0.36 | 1.63 |
| | 2.0 | 16.25 | 0.22 | 2.13 |
| | 2.6 | 18.68 | 0.26 | 2.49 |

The properties of the tablets obtained were particularly advantageous from the mechanical standpoint: even without precompression and for low compression forces, the cohesion is already highly satisfactory.

### EXAMPLE 5

During this test, a new active ingredient was used. The formulation remained identical to the one used in the previous example:

| Internal phase: | |
|---|---|
| Glyceryl Guiacol | 95.00% |
| Polyvinylpyrrolidone K90 ® | 2.23% |
| Starch 1551 ® | 1.48% |
| Silica | 0.15% |
| External phase: | |
| Ac-Di-Sol | 0.74% |
| Stearic acid | 0.25% |
| Colloidal silica | 0.15% |

A quantity of 1873.1 g of internal phase, corresponding to a quantity of 1800 g of glyceryl guiacol, was introduced into a share mixer. After mixing, a quantity of 208.1 g of distilled water was introduced into the mixer, namely a moisture content of 11.1% with respect to the dry mix. After extrusion through a 1 mm die, the product was dried and brought to a residual moisture content of 1.1%, then sized through an 800 μm screen.

After 21.55 g of external phase had been added, the granules were characterized in compression and their galenic properties were measured. The compression was carried out at a rate of 41 000 tablets per hour. The results of these tests are given in table VII hereinafter.

TABLE VII

| Forces (t) | | Hardness | Friability | Cohesion |
|---|---|---|---|---|
| PC | C | (kg) | (%) | (MPa) |
| 0.0 | 1.0 | 15.11 | 0.09 | 1.43 |
| | 1.5 | 16.75 | 0.13 | 1.67 |

Excellent mechanical properties were again found: without precompression and for very low compression forces, the cohesion of the tablets was highly satisfactory. It was of the same order of magnitude as the cohesion obtained with paracetamol under the same compression conditions.

This demonstrates the flexibility of the granulation technology. By comparing examples 4 and 5, it can be found that completely different active ingredients can be used with the same formulation and that the granules obtained give equivalent results in compression.

### EXAMPLE 6

During this test, the formulation used was as follows:

| Internal phase: | |
|---|---|
| Glyceryl guaiacol | 95.16% |
| Polyvinylpyrrolidone K30 ® | 3.00% |
| Maltodextrin MD040 | 1.00% |
| External phase: | |
| Stearic acid | 0.42% |
| Colloidal silica | 0.42% |

A quantity of 1875.6 g of internal phase, corresponding to a quantity of 1800 g of glyceryl guaiacol, was introduced into a share mixer. After mixing, a quantity of 208.4 g of distilled water was introduced into the mixer, namely a moisture content of 11.1% with respect to the dry mix. After extrusion through a 1 mm die, the product was dried and brought to a residual moisture content of 1.4%, then sized through an 800 μm screen.

After 15.9 g of external phase had been added, the granules were characterized in compression and their galenic properties were measured. The compression was carried out at a rate of 41 000 tablets per hour. The results of these tests are given in table VIII hereinafter.

TABLE VIII

| Forces (t) | | Hardness | Friability | Cohesion |
|---|---|---|---|---|
| PC | C | (kg) | (%) | (MPa) |
| 0.0 | 1.0 | 14.69 | 0.25 | 1.42 |
| | 1.5 | 14.59 | 0.49 | 1.44 |

Excellent mechanical properties are again found: without precompression and for very low compression forces, the cohesion of the tablets was already highly satisfactory.

This result once again illustrates the flexibility of the granulation technology. By comparing examples 5 and 6, it can be found that the nature of the active ingredient and the procedure are unchanged, but that the formulation is appreciably different: the way in which the granules behave in compression does, however, remain equivalent.

### EXAMPLE 7

In this test, use was made of ketoprofen by way of active ingredient. The formulation employed was as follows:

| Internal phase: | |
|---|---|
| Ketoprofen | 88.38% |
| Starch 1500 ® | 1.99% |
| Starch 1551 ® | 7.94% |
| Ac-Di-Sol | 0.99% |
| External phase: | |
| Mg stearate | 0.50% |
| Colloidal silica | 0.20% |

A quantity of 2247.1 g of internal phase, corresponding to a quantity of 2000 g of ketoprofen, was introduced into a share mixer. After mixing, a quantity of 493.3 g of distilled water was introduced into the mixer, namely a moisture content of 21.9% with respect to the dry mix. After extrusion through a 1 mm die, the product was dried and brought to a residual moisture content of 1.8%, then sized through an 800 μm screen.

After 15.8 g of external phase had been added, the granules were characterized in compression and their galenic properties were measured. The compression was carried out at a rate of 45 000 tables per hour. The results of these tests are given in table IX hereinafter.

TABLE IX

| Forces (t) | | Hardness | Friability | Cohesion | Disintegration |
|---|---|---|---|---|---|
| PC | C | (kg) | (%) | (MPa) | time(s) |
| 0.0 | 0.7 | 11.66 | 0.21 | 1.44 | 180 |
| | 1.0 | 12.49 | 0.15 | 1.68 | |
| | 1.6 | 14.86 | 0.28 | 1.91 | |
| | 2.0 | 13.95 | 0.16 | 1.83 | |

In this case too, the mechanical properties were highly satisfactory in that even without precompression and for very low compression, forces, the cohesion was highly satisfactory.

1. Method for formulating one or more active substance(s) into the form of granules that can be directly compressed, comprising wet granulation using a binder solution that binds the said active substance and, where appropriate, associated excipients, followed by the drying of the granules thus obtained, characterized in that the said granulation is carried out by low-pressure extrusion of the mix of active substance(s), binder solution and, where appropriate, excipient (s).

2. Method according to claim 1, characterized in that said granulation is carried out continuously.

3. Method according to claim 1, caracterized in that the low-pressure extrusion is carried out at a pressure of a 10⁶ Pa (10 bar) or less.

4. Method according to claim 1 or 2, characterized in that the extrusion is carried out at a pressure of the order of 3 to 4×10⁵ Pa (3 to 4 bar).

5. Method according to one of the preceding claims, characterized in that the extrusion is performed using a dome extruder, a basket extruder or a radial extruder.

6. Method according to one of the preceding claims, characterized in that extrusion is carried out using a dome extruder.

7. Method according to one of the preceding claims, characterized in that the binder solution is present at a content of 5% to 40% by weight of the dry mix active substances and excipients to be formulated.

8. Method according to one of the preceding claims, characterized in that the binder solution is based on water or on an aqueous solvent.

9. Method according to one of the preceding claims, characterized in that the binder solution contains a binding agent chosen from polyvinylpyrrolidone, cellulose, cellulose derivatives such as hydroxypropylmethylcellulose and hydroxypropylcellulose, natural gum, modified natural or synthetic gums such as gelatine, carob gum, guar gum, xanthan gum, alginates, carrageenans, maltodextrins and native or precooked starches.

10. Method according to one of the precedent claims, characterized in that the active substance to be transformed is present at 0,001% to 99,5% in weight of the total composition, the remainder being made up of the associated excipients.

11. Method according to one of the precedent claims, characterized in that the active substance is present if form of a powder, the particules of which have a median particle diameter of about 1 to 100 μm.

12. Method according to one of the precedent claims, characterized in that the active substance to be converted is a pharmaceutical active substance.

13. Method according to one of the preceding claims, characterized in that the active substance to be converted is N-aceytl-para-aminophenol.

14. Method according to claim 13, characterized in that the granules of N-aceytl-para-aminophenol obtained after drying have a residual moisture content of 2%±0.5%.

15. Method according to claim 13 or 14, characterized in that the N-aceytl-para-aminophenol is introduced in the form of granules with a residual moisture content other than 2%±0.5%.
